(19) United States
(12) Patent Application Publication
(10) Pub. No.: US 2016/0364503 A1
(43) Pub. Date: Dec. 15, 2016

(54) INDUSTRIAL PRODUCT DESIGN SYSTEM

(71) Applicant: HIROSHIMA UNIVERSITY, Higashi-Hiroshima-shi (JP)

(72) Inventors: Yuichi KURITA, Hiroshima (JP); Toshio TSUJI, Hiroshima (JP); Masaya KONDO, Hiroshima (JP); Yusuke KISHISHITA, Hiroshima (JP)

(73) Assignee: HIROSHIMA UNIVERSITY, Higashi-Hiroshima-shi (JP)

(21) Appl. No.: 15/246,282

(22) Filed: Aug. 24, 2016

Related U.S. Application Data
(63) Continuation of application No. PCT/JP2015/000721, filed on Feb. 17, 2015.

(30) Foreign Application Priority Data
Feb. 25, 2014 (JP) 2014-033795

(51) Int. Cl. G06F 17/50; A61B 5/11; A61B 5/0488; A61B 5/22

(52) U.S. Cl. CPC G06F 17/50; A61B 5/221; A61B 5/1118; A61B 5/0488

(57) ABSTRACT

An industrial product design system includes: a muscle activity acquisitor that acquires muscle activity required for each action of a given body area when a product user moves the body area to use an industrial product to be designed; a muscle activity normalizer that normalizes the acquired muscle activity; a function operator that calculates, as a design value change rate, mapping of the normalized muscle activity using a given function; and a design value corrector that corrects the design value of the industrial product to be designed with the design value change rate.

### CROSS-REFERENCE TO RELATED APPLICATIONS

This is a continuation of International Application No. PCT/JP2015/000721 filed on Feb. 17, 2015, which claims priority to Japanese Patent Application No. 2014-033795 filed on Feb. 25, 2014. The entire disclosures of these applications are incorporated by reference herein.

### BACKGROUND

The present disclosure relates to the design of industrial products, and more particularly to an industrial product design technique considering a feeling of kinetic burden by a product user.

Ergonomics that attempts to utilize physical and physiological features of human beings from an engineering standpoint is being actively applied to the development of human interfaces of various industrial products, among others. Ergonomics-based design is not only easy to use for human beings but also useful in preventing mistakes human beings are likely to make before they occur.

In ergonomics, a human body is represented by various models, and various types of motions of a human body are simulated by a model on a computer. Examples of such models include a finite element model as the most complicated one and a musculoskeletal model as a simple one where the framework, joints, and skeletal muscles of a human body are modeled. For example, as human body kinetic evaluation based on a musculoskeletal model, an evaluation system has been proposed by the present inventors, which, provided with evaluation indices for evaluating skills and sensibilities quantitatively, can automatically calculate a posture close to a posture of human beings (see Japanese Unexamined Patent

When a product user uses an industrial product, doing something such as pressing a button and operating a lever, the user moves his or her body area, thereby having a feeling of kinetic burden. When the feeling of kinetic burden is large, the user feels that the industrial product is hard to use. In reverse, when the feeling of kinetic burden is small, the user feels that the industrial product is easy to use.

The usability of an industrial product felt by the user is sometimes different with the difference of the body size of the user, etc. For example, an industrial product designed with a user having a standard body size in mind may be hard to use for a tall person or a short person. In other words, if the design of an industrial product fails to suit to the body size and muscle force of a user, the user will have a feeling of kinetic burden in a larger amount when using the product, thereby feeling that the product is hard to use. There is therefore a need for such design of an industrial product that will lighten the feeling of kinetic burden by the user. However, since the feeling of kinetic burden is a subjective matter for the user, it is difficult to deal with this sense quantitatively.

In the conventional industrial product design, in many cases, trial subjects have been asked to use trial products produced with various design values, and with fed-back opinions from the trial subjects, the design values have been changed. This method however requires a large amount of labor and time. Therefore, simpler industrial product design is desired.

### SUMMARY

The industrial product design system according to one aspect of the disclosure includes: a muscle activity acquisitor that acquires muscle activity required for each action of a given body area when a product user moves the body area to use an industrial product to be designed; a muscle activity normalizer that normalizes the acquired muscle activity; a function operator that calculates, as a design value change rate, mapping of the normalized muscle activity using a given function; and a design value corrector that corrects a design value of the industrial product to be designed with the design value change rate.

The “industrial product” as used herein refers to any of industrially mass-produced ones such as electric home appliances, computers, mobile terminals, and various mechanical products. The industrial product includes, not only the completed one of the product, but also various components thereof Also, the industrial product design may include arrangement of various types of objects operated by the user, such as buttons displayed on a touch panel screen, etc.

The “given body area” as used herein refers to an arm (superior limb), a finger, a foot (inferior limb), etc.

The “actions” as used herein may include moving a body area to various arrival points, subjecting a body area to reaching movement along various trajectories, moving a body area at various speeds, moving a body area under various load conditions, etc.

According to the above-described industrial product design system, a feeling of kinetic burden by the user during use of a product is evaluated by muscle activity, and the design value of the product to be designed is corrected based on the muscle activity.

### BRIEF DESCRIPTION OF THE DRAWINGS

The figures depict one or more implementations in accord with the present teachings, by way of example only, not by way of limitations. In the figures, the same reference numbers refer to the same or similar elements.

FIG. 1 is a functional block diagram of a main part of an industrial product design system according to an embodiment of the disclosure.

FIG. 2 is a view for explaining example actions of the user during use of a product.

FIG. 3 is a graph showing examples of muscle activity required for the actions shown in FIG. 2.

FIG. 4 is a graph obtained by normalizing the muscle activity shown in FIG. 3.

FIG. 5 is a graph showing a design value change rate obtained from the normalized muscle activity shown in FIG. 4.

FIG. 6 is a view showing an example of a keyboard designed by the industrial product design system according to the embodiment.

FIG. 7 is a view showing other examples of keyboards designed by the industrial product design system according to the embodiment.

FIG. 8 is a view showing an example of product design performed while presenting the user a muscle activity estimated value in real time.

FIG. 9 is a view showing another example of product design performed while presenting the user a muscle activity estimated value in real time.

### DETAILED DESCRIPTION

Embodiments are described in detail below with reference to the attached drawings. However, unnecessarily detailed description may be omitted. For example, detailed description of well known techniques or description of substantially the same elements may be omitted. Such omission is intended to prevent the following description from being unnecessarily redundant and to help those skilled in the art easily understand it.

The inventors provide the following description and the attached drawings to enable those skilled in the art to fully understand the present disclosure. Thus, the description and the drawings are not intended to limit the scope of the subject matter defined in the claims.

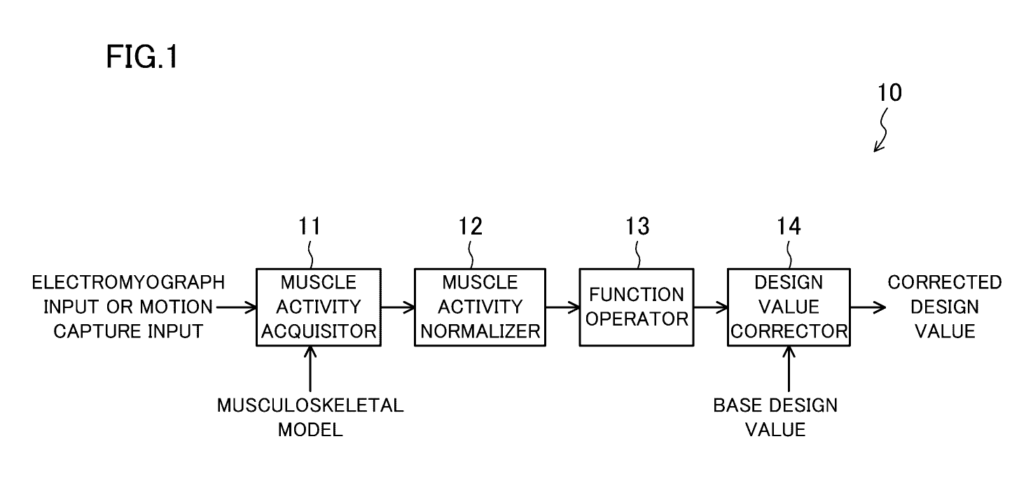

FIG. 1 is a functional block diagram of a main part of an industrial product design system 10 according to an embodiment of the disclosure. The industrial product to be designed may be any of industrially mass-produced ones such as electric home appliances, computers, mobile terminals, and various mechanical products. The industrial product includes, not only the completed one of the product, but also various components thereof. Also, the industrial product design may include arrangement of various types of objects operated by the user, such as buttons displayed on a touch panel screen, etc.

The industrial product design system 10 according to this embodiment includes a muscle activity acquisitor 11, a muscle activity normalizer 12, a function operator 13, and a design value corrector 14. The industrial product design system 10 can be implemented as dedicated hardware where the above components are comprised of semiconductor integrated circuits, etc. Alternatively, the above components may be described into instructions, and a general computer such as a PC may be made to execute the instructions stored in a non-transitory computer-readable medium, thereby implementing the system 10 on the general computer. Moreover, the industrial product design system 10 can be implemented by a combination of hardware and software.

The muscle activity acquisitor 11 acquires the muscle activity required for each action of a given body area when the product user moves the body area to use the industrial product to be designed. For example, the given body area may be a finger when the product to be designed is a keyboard used for a computer, etc., it may be a foot (inferior limb) when the product to be designed is a pedal of a bicycle, etc., and it may be an arm (superior limb) when the product to be designed is an automobile interior (a steering and seat arrangement, etc.), a touch panel, etc. Examples of the actions include moving the body area to various arrival points, subjecting the body area to reaching movement along various trajectories, moving the body area at various speeds, moving the body area under various load conditions, etc.

The muscle activity acquisitor 11 can acquire the muscle activity directly by measuring an electromyogram using an electromyograph. That is, a plurality of electrodes are stuck on the surface of a given body area of the user, to measure electromyograms during the maximum exertion of the voluntary muscle and during use of the product to be designed. The voluntary contraction strength (% MVC) obtained from the measurement results can be regarded as the muscle activity.

Alternatively, the muscle activity acquisitor 11 can acquire the muscle activity indirectly using a musculoskeletal model. More specifically, the muscle activity acquisitor 11 captures data of the motion and posture of the user who is using the product to be designed from a motion capture (not shown). The muscle activity acquisitor 11 then solves an inverse kinematic problem on the input motion capture data, thereby calculating the angle of each joint of the musculoskeletal model. The muscle activity acquisitor 11 also captures data of external load (external force). The muscle activity acquisitor 11 then solves an inverse kinematic problem from the calculated joint angle and the input external load data, thereby computing the moment of each joint of the musculoskeletal model. The thus-computed joint moment τ is generally expressed as Equation (1) below.

τ=M(q)\ddot{q}+C(q,\dot{q})+G(q)−E(q,\dot{q}) (1)

where, in Equation (1), M in the first term on the right-hand side represents the inertia force, C in the second term on the right-hand side represents the Coriolis force (centrifugal force), G in the third term on the right-hand side represents the gravity, and E in the fourth term on the right-hand side represents the external force. Also, q represents a generalized coordinate.

The muscle activity can be determined by performing static optimization from the above-computed joint moment. The relationship between the joint moment and the muscle activity is expressed by Equation (2) below.

$${\sum }_{m = 1}^{n} [ {α}_{m} f ( {F}_{m}^{0} , {t}_{m} , {v}_{m} ) ] {r}_{m , l} = {τ}_{j} \qquad ( 2 )$$

where n is the number of muscles, αₘ is the muscle activity, Fₘ⁰ is the isometric maximum muscle force, lₘ is the muscle length, vₘ is the muscle shortening velocity, f is a function having the isometric maximum muscle force, the muscle length, and the muscle shortening velocity as arguments, rⱼ is the moment arm, and τⱼ is the joint moment.

The muscle activity αₘ, indicating the degree of the activity of each muscle, takes on a value between 0 and 1. A value of the muscle activity αₘ closer to 1 indicates that the muscle is more activated.

It is said that human beings are unconsciously selecting such a motion that will make the muscle activity minimum. Therefore, at each moment of joint movement, by solving Equation (2) above so that the square sum of the muscle activity be minimum, or specifically, so that the object function J expressed by Equation (3) below be minimum, the muscle activity closer to the actual motion of human beings can be computed.

$$J = {\sum }_{m = 1}^{n} {( {α}_{m} )}^{2} \to min \qquad ( 3 )$$

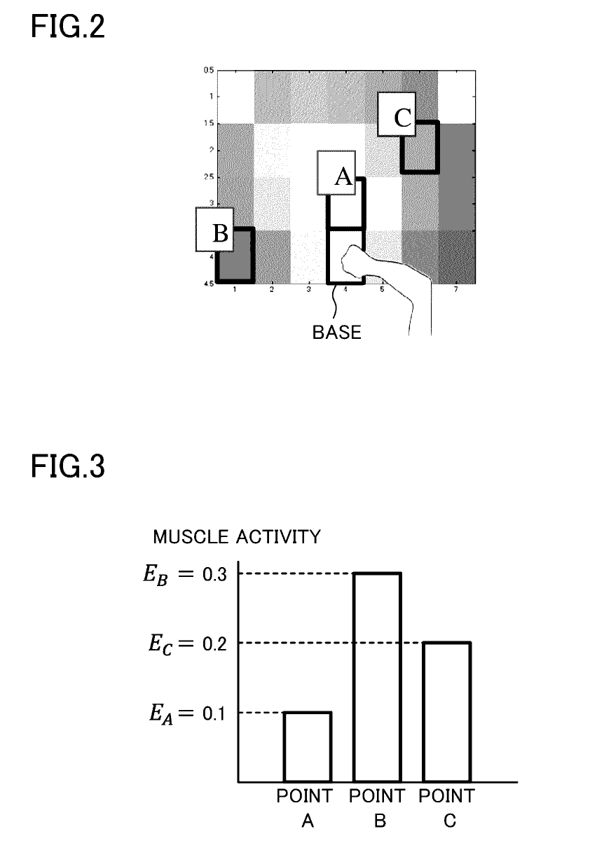

FIG. 2 is a view for explaining example actions of the user during use of a product. Assume, for example, that the product user moves his or her right hand from a base position (BASE) to points A, B, and C (arrival points) during use of the product to be designed. The muscle activity acquisitor 11 acquires the muscle activity of a muscle of the right upper arm required for such actions of the right hand (movements from the base position to points A, B, and C) directly or indirectly in a manner as described above. The muscle activity acquired by the muscle activity acquisitor 11 may be one of a typical muscle of the right upper arm, or an average of muscle activity values of the muscles of the right upper arm.

FIG. 3 is a graph showing examples of the muscle activity required for the actions shown in FIG. 2. For example, the muscle activity EA required for the action of moving the right hand from the base position to point A (hereinafter referred to as the “point A muscle activity) is 0.1, the muscle activity E_{B} required for the action of moving the right hand from the base position to point B (hereinafter referred to as the “point B muscle activity) is 0.3, and the muscle activity E_{C} required for the action of moving the right hand from the base position to point C (hereinafter referred to as the “point C muscle activity) is 0.2.

Referring back to FIG. 1, the muscle activity normalizer 12 normalizes the muscle activity acquired by the muscle activity acquisitor 11. The normalization can be performed according to Equation (4) below, for example.

$$= \frac{{E}_{X} - {E}_{MIN}}{{E}_{MAX} - {E}_{MIN}} \qquad ( 4 )$$

where E_{X} is the point X muscle activity, E_{MAX} is the maximum muscle activity, E_{MIN} is the minimum muscle activity, and E_{X} bar is the normalized point X muscle activity.

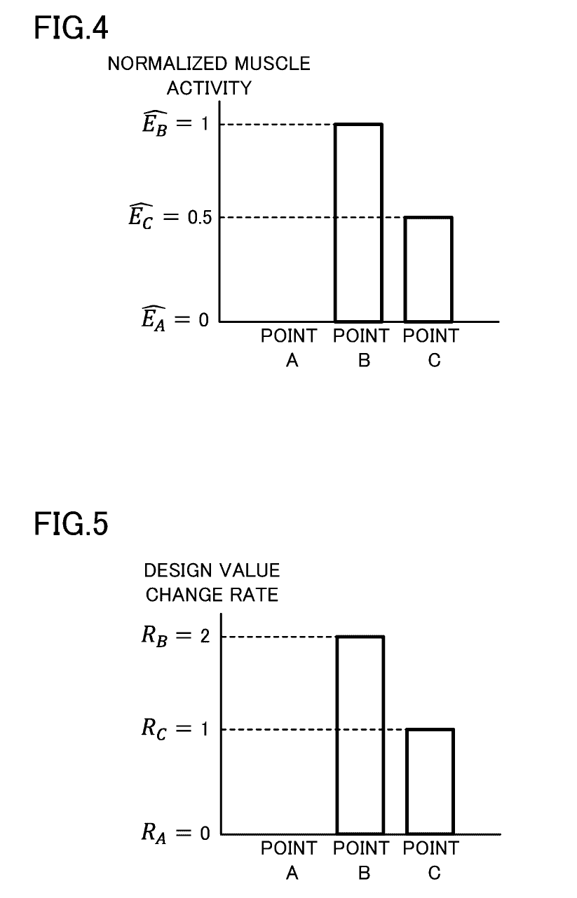

FIG. 4 is a graph obtained by normalizing the muscle activity shown in FIG. 3. In the example in FIG. 3, E_{MAX} is the point B muscle activity E_{B}, and E_{MIN} is the point A muscle activity E_{A}. The normalized point A muscle activity E_{A} bar (minimum muscle activity) is 0, and the normalized point B muscle activity E_{B} bar (maximum muscle activity) is 1. The normalized point C muscle activity E_{C} bar is 0.5 that is a value between 0 and 1.

Referring back to FIG. 1, the function operator 13 calculates, using a given function, mapping of the muscle activity normalized by the muscle activity normalizer 12, and sets the results as design value change rates. As will be described later, the design value change rate is a coefficient for correcting the design value of each part of a product to be designed. For example, the design value change rate R_{X} at point X of a product to be designed can be calculated according to Equation (5) below.

R_{X}=ƒ( [symbol] ) (5)

where f(Z) is the function of Z. As the function f, the linear function (f(Z)=a·Z), the exponential function (f(Z)=a·e^{b·Z}), the logarithmic function (f(Z)=a·log Z), etc. can be used. Which function to use can be determined according to the product to be designed.

FIG. 5 is a graph showing the design value change rate obtained from the normalized muscle activity shown in FIG. 4. In the example in FIG. 5, the linear function is used as the function f.

Referring back again to FIG. 1, the design value corrector 14 corrects the design values of the product to be designed with the design value change rates. For example, correction of a design value can be performed according to Equation (6) below.

I_{X}I_{BASE}+I_{S}·R_{X} (6)

where I_{BASE} is the design base value of the product to be designed (e.g., the design value at the base position shown in FIG. 2), I_{S} is the design change base value of the product to be designed, and I_{X} is a corrected design value at point X of the product to be designed.

The design value includes at least one of the position and color of each part of the industrial product to be designed, the reaction force of the part against operation, the characteristic of the vibration of the part during the operation, and the contact detection sensitivity. For example, when the product to be designed is a mechanical keyboard, the design value includes the height and position, the color (brightness, chroma, hue, etc.), the magnitude of the reaction force, the hardness of a spring, etc., of each button. Also, when the product to be designed is a touch panel keyboard, the design value may also include the vibration characteristic (frequency, amplitude, vibrating time, etc.) at the pressing of each button, the sensitivity as to how much contact strength is required to detect the contact (contact detection sensitivity), etc.

### EXAMPLE

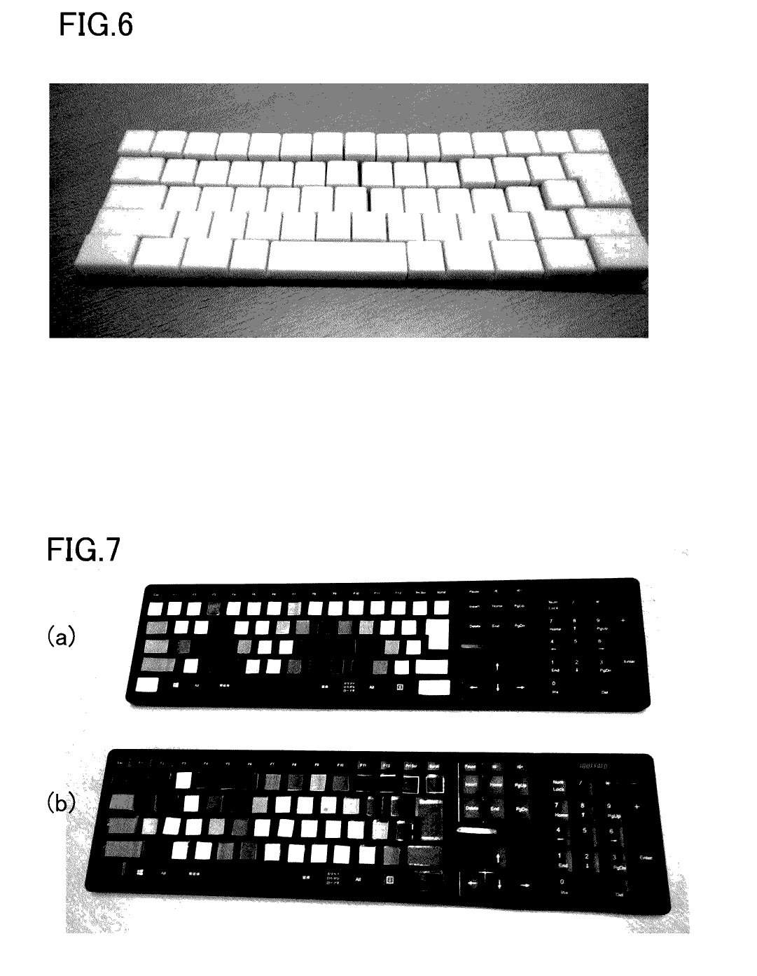

FIG. 6 shows an example of a keyboard designed by the industrial product design system 10. The muscle activity is comparatively large when buttons in the first row, the Enter key etc., which are located apart from the home position, are pressed. Therefore, these buttons are made a little higher in height than the standard value. With this arrangement, buttons at positions difficult to reach for pressing from the home position become easy to press, whereby the feeling of kinetic burden by the product user can be lightened.

FIG. 7 shows other examples of keyboards designed by the industrial product design system 10. The upper part (a) of FIG. 7 shows an example where, with the standard color of buttons being black, buttons larger in design value change rate are made closer to white. For human beings, white ones appear more rising upward than black ones. Therefore, buttons at positions difficult to reach for pressing from the home position are made white, whereby the feeling of kinetic burden by the product user can be lightened from the visual standpoint. The lower part (b) of FIG. 7 shows an example where, with the standard color of buttons being white, buttons larger in design value change rate are made closer to black. Human beings perceive black ones as being lighter than white ones. Therefore, buttons at positions difficult to reach for pressing from the home position are made black, whereby the feeling of kinetic burden by the product user can be lightened from the visual standpoint.

As described above, according to this embodiment, a feeling of kinetic burden by a product user is evaluated quantitatively based on objective indices, to permit industrial product design considering a feeling of kinetic burden. Thus, various industrial products can be customized to suit to the body size and muscle strength of the product user.

In designing a product using the industrial product design system 10 according to this embodiment, the muscle activity of each body area calculated (estimated) by the muscle activity acquisitor 11 may be presented to the user (in this case, the person who designs the product using the industrial product design system 10) in real time. More specifically, a muscle activity estimated value may be superimposed on an image of the user shot by a camera and displayed on a monitor, using augmented reality (AR) technology. The user can design the product while viewing the muscle activity estimated value-superimposed image.

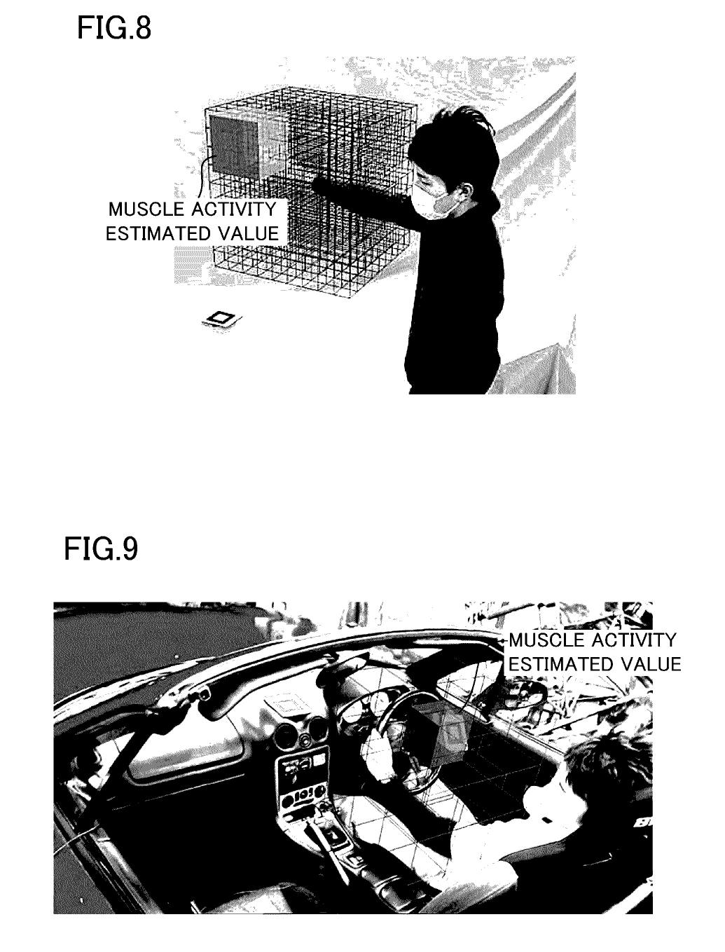

FIG. 8 is a view showing an example of product design performed while presenting the user a muscle activity estimated value in real time. In a case of designing a product, such as a control board and a control panel, which the product user operates with his or her hand by moving his or her upper arm, for example, the calculation result (estimated value) of the muscle activity may be displayed at the position of the hand of the user, as shown in FIG. 8. FIG. 9 is a view showing another example of product design performed while presenting the user a muscle activity estimated value in real time. In a case of determining an optimal seat position of an automobile, for example, the calculation result (estimated value) of the muscle activity may be displayed at the position of a hand of the user who is seated in the automobile holding the steering wheel, as shown in FIG. 9.

In either of the examples in FIGS. 8 and 9, where the muscle activity changes with the motion of the upper arm of the user, the muscle activity estimated value may be presented with change of the color. In this way, by presenting the muscle activity obtained by the muscle activity acquisitor 11 to the user in real time, the industrial product design can be made easier.

Various embodiments have been described above as example techniques of the present disclosure, in which the attached drawings and detailed description are provided.

As such, elements illustrated in the attached drawings or the detailed description may include not only essential elements for solving the problem, but also non-essential elements for solving the problem in order to illustrate such techniques. Thus, the mere fact that those non-essential elements are shown in the attached drawings or the detailed description should not be interpreted as requiring that such elements be essential.

Since the embodiments described above are intended to illustrate the techniques in the present disclosure, it is intended by the following claims to claim any and all modifications, substitutions, additions, and omissions that fall within the proper scope of the claims appropriately interpreted in accordance with the doctrine of equivalents and other applicable judicial doctrines.

What is claims is:

1. An industrial product design system that designs an industrial product, comprising:
a muscle activity acquisitor that acquires muscle activity required for each action of a given body area when a product user moves the body area to use an industrial product to be designed;
a muscle activity normalizer that normalizes the acquired muscle activity;
a function operator that calculates, as a design value change rate, mapping of the normalized muscle activity using a given function; and
a design value corrector that corrects a design value of the industrial product to be designed with the design value change rate.

2. The industrial product design system of claim 1, wherein
the muscle activity acquisitor computes the muscle activity required for each action of the given body area based on a musculoskeletal model of the product user.

3. The industrial product design system of claim 1, wherein
the design value includes at least one of a position and color of each part of the industrial product to be designed, a reaction force of the part against operation, a characteristic of vibration of the part during operation, and a contact detection sensitivity of the part during operation.

4. The industrial product design system of claim 2, wherein
the design value includes at least one of a position and color of each part of the industrial product to be designed, a reaction force of the part against operation, a characteristic of vibration of the part during operation, and a contact detection sensitivity of the part during operation.

5. An industrial product design method for designing an industrial product using a computer, comprising:
acquiring muscle activity required for each action of a given body area when a product user moves the body area to use an industrial product to be designed;
normalizing the acquired muscle activity;
calculating, as a design value change rate, mapping of the normalized muscle activity using a given function; and
correcting a design value of the industrial product to be designed with the design value change rate.

6. The industrial product design method of claim 5, wherein
the acquiring muscle activity includes computing the muscle activity required for each action of the given body area based on a musculoskeletal model of the product user.

7. The industrial product design method of claim 5, wherein
the design value includes at least one of a position and color of each part of the industrial product to be designed, a reaction force of the part against operation, a characteristic of vibration of the part during operation, and a contact detection sensitivity of the part during operation.

8. The industrial product design method of claim 6, wherein
the design value includes at least one of a position and color of each part of the industrial product to be designed, a reaction force of the part against operation, a characteristic of vibration of the part during operation, and a contact detection sensitivity of the part during operation.

9. A non-transitory computer-readable medium with instructions stored therein, the instructions, when executed by a data processing system, case the data processing system to perform a method of designing an industrial product, the method comprising:
acquiring muscle activity required for each action of a given body area when a product user moves the body area to use an industrial product to be designed;
normalizing the acquired muscle activity;
calculating, as a design value change rate, mapping of the normalized muscle activity using a given function; and
correcting a design value of the industrial product to be designed with the design value change rate.

10. The non-transitory computer-readable medium of claim 9, wherein
the acquiring muscle activity includes computing the muscle activity required for each action of the given body area based on a musculoskeletal model of the product user.

11. The non-transitory computer-readable medium of claim 9, wherein
the design value includes at least one of a position and color of each part of the industrial product to be designed, a reaction force of the part against operation, a characteristic of vibration of the part during operation, and a contact detection sensitivity of the part during operation.

12. The non-transitory computer-readable medium of claim 10, wherein
the design value includes at least one of a position and color of each part of the industrial product to be designed, a reaction force of the part against operation, a characteristic of vibration of the part during operation, and a contact detection sensitivity of the part during operation.
